(19) United States
(12) Patent Application Publication
(10) Pub. No.: US 2024/0108610 A1
(43) Pub. Date: Apr. 4, 2024

(54) RECTAL ADMINISTRATION OF ROFLUMILAST FOR THE TREATMENT OF CROHN'S DISEASE

(71) Applicant: King Abdulaziz University, Jeddah (SA)

(72) Inventors: Hussam Aly Sayed Murad, Jeddah (SA); Abdulkader Mohammed Shaikh-Omar, Jeddah (SA); Marwan A. Bakarman, Jeddah (SA); Thamer Mohammed Alqurashi, Jeddah (SA)

(21) Appl. No.: 18/243,740

(22) Filed: Sep. 8, 2023

Related U.S. Application Data
(60) Provisional application No. 63/411,202, filed on Sep. 29, 2022.

(51) Int. Cl. A61K 31/44; A61K 9/00; A61K 9/02; A61K 31/655; A61P 1/06

(52) U.S. Cl. CPC A61K 31/44; A61K 9/0031; A61K 9/02; A61K 31/655; A61P 1/06

(57) ABSTRACT

A method for treating Crohn's Disease in a subject in need thereof is provided. The method includes administering rectally to the subject a therapeutically effective amount of a composition comprising roflumilast. The composition may be administered repetitively over a period of days and may contain or be co-administered with one or more agents different from roflumilast that are directed against bowel disease. A suppository dosage form comprising roflumilast and a pharmaceutically acceptable carrier are also provided.

### CROSS-REFERENCE TO RELATED APPLICATIONS

This application claims benefit of U.S. provisional patent application 63/411,202, filed Sep. 29, 2022, the complete contents of which is hereby incorporated by reference.

### FIELD OF THE INVENTION

The invention is generally related to the use of roflumilast for the treatment of Crohn's disease via rectal delivery.

### BACKGROUND OF THE INVENTION

The prevalence of Crohn's disease (CD), a predominant type of inflammatory bowel disease (IBD), has been increasing over the last decades. CD is a chronic, idiopathic, immunologically mediated disease that is triggered in a genetically predisposed person by multiple environmental factors (1). CD occurs as a patchy granuloma affecting any part of the gastrointestinal tract, especially the ileum and first part of the colon. It involves the entire bowel wall and manifests as abdominal masses and perianal lesions. Numerous anti-inflammatory or immunomodulatory treatments, including sulfasalazine and corticosteroids, are available for CD; however, they have many side effects and variable efficacy, especially in active CD (2). Thus, it is necessary to search for new medications for CD and, to be relevant to human CD, the animal model should be already established, chronic, and immune-mediated (3). Trinitrobenzene sulfonic acid (TNBS) induces an experimental model of colitis with a T helper 1 (Th1) immune pattern that is similar to human CD, but with certain limitations (4).

Phosphodiesterase IV (PDE4) inhibitors have anti-inflammatory effects that are rather similar to those of corticosteroids yet with the advantage of not interfering with the hypothalamo-pituitary-adrenal axis. Roflumilast, a highly selective oral PDE4 inhibitor with anti-inflammatory and anti-fibrotic effects, increases intracellular cyclic adenosine monophosphate and, hence, reduces the production of numerous inflammatory mediators such as tumor necrosis factor-α (TNF-α) and interleukins (ILs). It is the first licensed member of this class and has been approved by the FDA as an add-on treatment for chronic obstructive pulmonary disease. Moreover, in ovalbumin-induced asthmatic mice, inhaled roflumilast substantially improved inflammation and histopathological changes, as it decreased numbers of neutrophils, eosinophils, and macrophages in the bronchoalveolar lavage fluid. Thus, inhaled roflumilast could be a useful off-label treatment for neutrophilic and eosinophilic asthma (5). Oral roflumilast may cause weight loss, headache, nausea, emesis, and diarrhea. Its bioavailability is nearly 80%, and consuming food delays, but does not decrease, its absorption. Thus, taking roflumilast with food could improve its gastrointestinal side effects (6).

In view of the above-described issues, alternative dosage forms and delivery methods for roflumilast are needed.

### SUMMARY

An aspect of the disclosure provides a method of treating Crohn's Disease in a subject in need thereof comprising administering rectally to the subject a therapeutically effective amount of a composition comprising roflumilast. In some embodiments, the composition is administered repetitively over a period of days, for example once daily for 1-30 days. In some embodiments, the therapeutically effective amount is 1-10 mg/kg. In some embodiments, the composition contains or is co-administered with one or more agents different from roflumilast, wherein said one or more agents are directed against bowel disease. In some embodiments, the one or more agents includes a phosphodiesterase IV inhibitor. In some embodiments, the one or more agents includes sulfasalazine.

Another aspect of the disclosure provides a suppository dosage form comprising roflumilast and a pharmaceutically acceptable carrier.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

FIGS. 1A-C. The effects of roflumilast in trinitrobenzenesulfonic acid (TNBS)-induced colitis in rats. (A) Disease activity score: ^{a}P<0.001 SS & OR & RR vs NC and PC, ^{b}P<0.01 OR vs SS (P=0.003), ^{c}P<0.001 RR vs SS, ^{d}P<0.05 RR vs OR (P=0.037). (B) Colon length: ^{e}P<0.001 OR & RR vs NC, ^{f}P<0.01 SS vs NC (=0.001), ^{g}P<0.05 RR vs PC (=0.023), ^{k}P<0.001 SS & OR vs PC, ^{m}P<0.01 RR vs OR (=0.006). (C) Colonic macroscopic appearance. The scale bar indicates 1 cm: The NC group shows a normal appearance of the colon. The PC group shows severe colonic injury characterized by inflammation, mucosal edema, and thickening of the bowel wall. The SS group shows marked improvement, the OR group shows moderate to marked improvement, and the RR group shows mild improvement. Data in (A) and (B) are reported as means±SE; one-way ANOVA and Tukey's post hoc test. NC: normal control; PC: positive control; SS: sulfasalazine; OR: oral roflumilast; RR: rectal roflumilast.

FIGS. 2A-E. Photomicrographs of rat colonic sections stained with H&E (×20, scale bar 100 μm). (A) The normal control group shows normal mucosal layers, intact surface epithelium (arrows), normally shaped crypts with numerous goblet cells (arrow), and narrow connective tissue submucosa (stars). Tunica submucosa (s) consisting of loose connective tissue rich in blood vessels and lymph vessels, and muscularis mucosae (m) consisting of inner circular and outer longitudinal smooth muscle fibers are seen. (B) The positive control group shows loss of surface crypt epithelium (arrows) and destruction of crypts that appear darker with less goblet cells (d). The regions of the lost crypts are replaced by inflammatory infiltrate and fibrous tissue (stars). Thickened fibrosed muscularis muscle and widened submucosa due to edema (star) are seen. (C) The sulfasalazine group shows marked improvement with nearly normal appearance of colonic surface (thin arrows) and crypts (thick arrows) with few desquamated cells, especially colonocytes. (D) The oral roflumilast group shows moderate improvement with focal loss of surface crypt cells (arrows) and some lost crypts (stars). Other crypts appear normal with numerous goblet cells (arrows). The submucosa is somewhat edematous (star) with congested blood vessels (c). The colonic wall displays thickening with patches of fibrosis and epithelial desquamation (d). (E) The rectal roflumilast group shows mild improvement with apparent thickening of the colonic mucosa with numerous patches of crypt loss (stars) and dark degenerated surface cells (thin arrows). The crypts appear deformed, degenerated, and dark, with few goblet cells (thick arrows). Desquamated colonic cells (d), especially the cells lining the crypts, and numerous congested blood vessels in the propria submucosa are seen.

FIGS. 3A-E. Photomicrographs of rat colonic sections stained with Masson trichrome (×20, scale bar 100 μm). (A) The normal control group shows colonic mucosa with normal crypts and goblet cells (arrows) separated by scanty loose, stained collagen (thin arrows). (B) The positive control group shows a marked increase in collagen deposition (dotted arrows) replacing the degenerated crypts (thick arrows) and around the congested and thickened blood vessels (c) in the submucosa. (C) The sulfasalazine group shows a marked decrease in collagen (dotted arrows) deposition between crypts (arrow). Some crypts of Lieberkühn showed ulceration and leucocyte accumulation. Some congested blood vessels (c) in the propria submucosa and numerous goblet cells (g) are seen. (D) The oral roflumilast group shows a moderate decrease in collagen fibers (arrows) between colonic crypts (arrow) and in submucosa. Numerous goblet cells (g) and some congested blood vessels are seen between the crypts of Lieberkühn. (E) The rectal roflumilast group shows a mild decrease in collagen deposition (arrows) between colonic crypts (arrow), ulcerated crypts of Lieberkühn, and congested blood vessels (c) with leucocyte accumulation.

FIGS. 4A-E. Photomicrographs of rat colonic sections stained with PAS (×20, scale bar 100 μm). (A) The normal control group shows colonic mucosa with normal crypts and numerous PAS-stained goblet cells (arrows). (B) The positive control group shows a marked decrease or complete loss of PAS-stained goblet cells (dotted arrows). (C) The sulfasalazine group shows a nearly normal population of PAS-stained goblet cells (arrows). (D) The oral roflumilast group shows a moderate population of PAS-stained goblet cells (arrows). (E) The rectal roflumilast group shows a small population of PAS-stained goblet cells (arrows) indicating faint positive PAS reactions with patches of decrease or complete loss (dotted arrows).

FIGS. 5A-E. Photomicrographs of rat colonic sections stained for KI-67 immunoexpression in the mucosa (×20, scale bar 100 μm). (A) The normal control group shows strong positive reactions indicating extensive immunoexpression in crypt cells (arrow) and surface cells (arrows). (B) The positive control group shows very faint positive reactions indicating very mild immunoexpression in crypt cells (thick arrows) and surface cells (thin arrows). (C) The sulfasalazine group shows moderately positive reactions indicating moderate immunoexpression in crypt cells (arrow) and surface cells (arrows). (D) The oral roflumilast group shows mildly positive reactions indicating weak immunoexpression in crypt cells (arrow) and surface cells (arrows). (E) The rectal roflumilast group shows very mild positive reactions indicating very weak immunoexpression in crypt cells (arrow) and surface cells (arrows).

FIGS. 6A-E. Photomicrographs of rat colonic sections stained for TNF-α immunoexpression in the mucosa (×20, scale bar 100 μm). (A) The normal control group shows negative reactions in the crypt cells and goblet cells (arrow), except for a few cells in the connective tissue lamina (dotted arrow). (B) The positive control group shows highly positive reactions in the degenerated crypt cells (arrow) and inflammatory cells between the crypts (dotted arrows). (C) The sulfasalazine group shows very faint positive reactions in the surface cells (arrow) and crypt cells (arrow). (D) The oral roflumilast group shows moderately positive reactions in the surface cells (dotted arrows) and crypt cells (arrow). (E) The rectal roflumilast group shows strong positive reactions in crypt cells (arrow) and degenerated cells (dotted arrows).

### DETAILED DESCRIPTION

Embodiments of the disclosure provide suppository dosage forms of roflumilast and methods of treating Crohn's disease in a subject in need thereof. It is demonstrated in the Example herein that rectally-delivered roflumilast significantly improves TNBS-induced chronic colitis (a model for Crohn's disease) in rats.

Roflumilast (e.g. commercially available as Daliresp® and Zoryve®) is a selective phosphodiesterase-4 (PDE4) inhibitor often used to treat chronic obstructive pulmonary disease (via oral administration) and plaque psoriasis (via topical administration). PDE4 is a cyclic-3′,5′-adenosinemonophosphate (cyclic AMP, cAMP)-metabolizing enzyme expressed on nearly all immune and pro-inflammatory cells, in addition to structural cells like those of the smooth muscle or epithelium. The resultant increase in intracellular cAMP induced by roflumilast's inhibition of PDE4 is thought to mediate its disease-modifying effects.

The rectal administration of roflumilast as described herein avoids the side effects associated with the oral route and provides a useful therapy for Crohn's disease. It exerts anti-diarrheal and anti-spasmodic effects by inhibiting PDE4 and voltage-gated Ca⁺⁺ channels at low doses.

The term “roflumilast” as used herein includes roflumilast free base, its pharmacologically active metabolites including roflumilast-N-oxide, its pharmaceutically acceptable salt or its polymorphs. Roflumilast present in the composition according to present disclosure can be in crystalline form or amorphous form.

A composition containing roflumilast may contain pharmaceutically acceptable carriers or excipients. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In some embodiments, the composition is in the form of an enema, a foam, an ointment, a cream, a gel, a lotion, a paste, or a suppository. A suppository may be prepared by mixing the roflumilast with one or more suitable nonirritating excipients or carriers comprising, for example, cocoa butter, polyethylene glycol, a suppository wax or a salicylate, which is solid at room temperature, but liquid at body temperature and, therefore, will melt in the rectum and release the active agent. In one embodiment, the composition is formulated as a liquid or semisolid. In some embodiments, the composition is formulated as a solution or a suspension in an aqueous or non-aqueous liquid, or as an oil-in-water or water-in-oil liquid emulsion.

The preparation of pharmacologically suitable compositions for use as medicaments is well known to those of skill in the art. Typically, such compositions are prepared either as liquid solutions or suspensions, however solid dry forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. If it is desired to administer an oral form of the composition, various thickeners, flavorings, diluents, emulsifiers, dispersing aids or binders and the like may be added. The composition of the present disclosure may contain any such additional ingredients so as to provide the composition in a form suitable for administration. The final amount of compound in the formulations may vary. However, in general, the amount in the formulations will be from about 1% to about 99%, e.g. from about 5% to about 70%, or from about 10% to about 30%.

The compositions of the present disclosure may also contain other components such as, but not limited to, additives, adjuvants, buffers, tonicity agents, and preservatives. In any of the compositions of this disclosure, the mixtures are preferably formulated at about pH 5 to about pH 8. This pH range may be achieved by the addition of buffers to the composition. It should be appreciated that the compositions of the present disclosure may be buffered by any common buffer system such as phosphate, borate, acetate, citrate, carbonate and borate-polyol complexes, with the pH and osmolality adjusted in accordance with well-known techniques to proper physiological values.

The compositions described herein are useful for the treatment of diseases that can be ameliorated by an anti-inflammatory agent, such as inflammatory bowel disease, (e.g., Crohn's and colitis). One embodiment provides a method of treating Crohn's Disease in a subject in need thereof comprising administering rectally to the subject a therapeutically effective amount of a composition comprising roflumilast. Other suitable administration routes include, but are not limited to: by injection, inhalation, orally, intravaginally, intranasally, by ingestion of a food or product containing the compound, topically, as eye drops, via sprays, etc. The methods described herein may ameliorate, reduce, or inhibit one or more symptoms of the disease, such as inflammation of the digestive tract (i.e. the small or large intestine, e.g. the colon), abdominal pain, diarrhea, fatigue, weight loss, and malnutrition.

A patient or subject to be treated by any of the compositions or methods of the present disclosure can mean either a human or a non-human animal including, but not limited to dogs, horses, cats, rabbits, gerbils, hamsters, rodents, birds, aquatic mammals, cattle, pigs, camelids, and other zoological animals.

In some embodiments, the compound is administered to the subject in a therapeutically effective amount. By a “therapeutically effective amount” is meant a sufficient amount of active agent to treat the disease or disorder at a reasonable benefit/risk ratio applicable to any medical treatment. It will be understood that the total daily usage of the compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. For example, it is well within the skill of the art to start doses of the compound at levels or frequencies lower than those required to achieve the desired therapeutic effect and to gradually increase the dosage or frequency until the desired effect is achieved. However, the daily dosage of the active agent may be varied over a wide range from 0.01 to 1,000 mg per adult per day. In particular, the compositions contain 0.01, 0.05, 0.1, 0.5, 1, 5, 10, 20, 30, 40, 50, 60, 70, 80, 90, 100, 150, 200, 250, 300, 350, 400, 450, 500, 550, 600, 650, 700, 750, 800, 850, 900, 950, or 1000 mg of the compound, including all multiples of 5 and 10 between 0.01 and 1000 (e.g. 100, 105, 110, 115, etc.). An effective amount of the drug is ordinarily supplied at a dosage level from 0.0002 mg/kg to about 20 mg/kg of body weight per day, especially from about 1 mg/kg to 10 mg/kg of body weight per day. In some embodiments, the composition is administered repetitively over a period of days, for example once daily for 1-30 days.

In some embodiments, the composition contains or is co-administered with one or more agents different from roflumilast, wherein said one or more agents are directed against bowel disease. In some embodiments, the one or more agents includes a phosphodiesterase IV inhibitor. In some embodiments, the one or more agents includes sulfasalazine. Other agents such as additional anti-inflammatory agents, steroids, antibiotics, anti-fungal agents, analgesics, or anti-neoplastic agents may also be included or co-administered with the composition. For clarity, any additional agents may be administered in a single dosage form with roflumilast or in separate dosage forms.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### Example

### Summary

Roflumilast, a highly selective oral phosphodiesterase IV inhibitor, exerts anti-inflammatory and anti-fibrotic effects. Oral roflumilast causes gastrointestinal side effects, especially vomiting, which could be reduced by administering roflumilast via off-label routes. Inhaled roflumilast reportedly improved inflammatory and histopathological changes in asthmatic mice. The current study investigated the effects of oral and rectal roflumilast on trinitrobenzenesulfonic acid (TNBS)-induced chronic colitis in rats, an experimental model resembling human Crohn's disease. Five groups of rats (n=8) were used: normal control, TNBS-induced colitis, and three TNBS-treated colitic groups, which received oral sulfasalazine (500 mg·kg⁻¹·day⁻¹), oral roflumilast (5 mg·kg⁻¹·day⁻¹), or rectal roflumilast (5 mg·kg⁻¹·day⁻¹) for 15 days after colitis induction. Then, the following were assessed: the colitis activity score, tumor necrosis factor (TNF)-α, interleukin (IL)-2, and IL-6 serum levels, colonic length, and myeloperoxidase, malonaldehyde, and glutathione levels. Histological examinations employed H&E, Masson trichrome, and PAS stains in addition to immunostaining for KI-67 and TNF-α. The TNBS-induced colitis rats showed significant increases in disease activity scores, serum TNF-α, IL-2, and IL-6 levels, and colonic myeloperoxidase and malonaldehyde content. They also showed significant decreases in colonic length and glutathione levels in addition to histopathological and immunohistochemical changes. All the treatments significantly improved all these changes. Sulfasalazine provided the greatest improvement, followed by oral roflumilast, and then rectal roflumilast. In conclusion, both oral and rectal roflumilast improved TNBS-induced chronic colitis, demonstrating the use of roflumilast as an additional treatment for Crohn's disease.

### Materials and Methods

### Animals

This study was approved by the Ethics Committee at King Abdulaziz University (No. 499/19) and adhered to the international guidance for use of laboratory animals. Chemicals and drugs were purchased from Sigma-Aldrich Corp. (USA) unless mentioned otherwise. Female Sprague-Dawley rats (180-200 g) were housed in cages at 22° C. with a 12-h light/dark cycle. Rats were acclimatized for 7 days before starting the procedures. Food and water were available ad libitum.

### Induction of Colitis and Treatment Groups

Chronic colitis was induced by weekly rectal injections of increasing doses of TNBS over 6 weeks (15, 30, 45, 60, 60, and 60 mg from day 0 to day 35), as previously described (4,12). Briefly, after food deprivation for 24 h, rats were anesthetized with an intraperitoneal injection of 1% isoflurane. TNBS was dissolved in ethanol (50%) and instilled into the colon using a cannula (0.25 mL), and then the rats were held upside down for 3 min to prevent escape of the intracolonic TNBS. Roflumilast was dissolved in dimethyl sulfoxide and diluted in 0.5% carboxymethyl cellulose sodium to a final concentration of 5.0 mg·kg⁻¹·day⁻¹ as previously described (13). Five groups of rats (n=8) were established: normal control (NC group), TNBS-treated (TNBS group, positive control), and three TNBS-treated groups that received oral sulfasalazine (SS group, 500 mg/kg) (14), oral roflumilast (by gastric gavage, OR group, 5 mg/kg), or rectal roflumilast (RR group, 5 mg/kg). Treatments were administered once daily in a volume of 200 μL for 15 days starting 48 h after the induction of colitis (from day 37) (15), and the control rats received equal amounts of vehicle. At the end of the treatment period, the rats were evaluated with the colitis activity score. To detect the effects of the treatments on systemic inflammatory processes, blood was collected for measurements of the proinflammatory mediators TNF-α, IL-2, and IL-6. At the end of the 15-day treatment period, the rats were sacrificed by cervical dislocation, and the colon was isolated, cleaned of feces, and measured for weight and length. Colonic myeloperoxidase (MPO) activity and glutathione (GSH) and malonaldehyde (MDA) levels were measured. The colon was examined macroscopically for gross changes and microscopically in sections stained with hematoxylin and eosin (H&E), Masson trichrome, and Periodic Acid Schiff (PAS) stains as well as KI-67 and TNF-α immunostaining.

### Disease Activity Score

The colitis activity index was determined to evaluate the activity of intestinal inflammation as previously described with a slight modification (16). The combined score was calculated based on the following parameters: i) weight loss (0: no loss; 1: 1-15%; 2: >15%); ii) blood in feces (0: no blood; 1: traces of blood (≤50% of surface); 2: gross bleeding (>50% of surface); and iii) consistency of feces (0: ordinary; 1: loose stools; 2: diarrhea).

### Colonic Macroscopic Examination

After the rats were sacrificed, their colon was excised, and its length was measured. The appearance of the colon regarding edema, ulceration, and necrosis was evaluated (17).

### Serum Measurements

Blood was collected through cardiac puncture. The serum was obtained and stored at −80° C. until analysis. The measurements were done using ELISA kits for TNF-α, IL-2, and IL-6 (Innovative Research, USA), according to the manufacturer's guidelines.

### Colonic Measurements

The levels of MPO (a marker of neutrophil activity), GSH (one of the most important cellular antioxidants), and MDA (an indicator of lipid peroxidation) in colonic tissue homogenates were measured using the commercially available kits as previously described: MPO ELISA kit (Innovative Research) (18), GSH colorimetric assay kit (Innovative Research) (19), and MDA ELISA kit (Neobio Lab Comp., USA) (20).

### Histopathological Examination

The colonic segments were fixed in 10% buffered formalin and embedded in paraffin. Afterwards, 3-5-μm-thick sections were made and stained with H&E, Masson's trichrome for collagen, and PAS for lymphocytes and mucopolysaccharides. The severity of the lesions was evaluated by a blinded histopathologist as normal, mild, moderate, or marked injury (21).

### Immunohistochemical Examination

The colonic sections were immunohistochemically stained with KI-67 and TNF-α. For KI-67 immunostaining, the avidin biotin peroxidase method was used as previously described (22). Briefly, the colonic sections were mounted on charged slides, deparaffinized, and washed in buffered phosphate-buffered saline (PBS) (pH 7.2) for 5 min. The sections were incubated with antisera with the specific KI-67 primary antibody at 1:80 dilution (Dako, M7240, USA) in a humidified chamber at room temperature overnight. Afterwards, the slides were washed in PBS, incubated with a horseradish peroxidase polymer for 15 min at room temperature, washed again in PBS, and incubated in diaminobenzidine as chromogen. The sections were then stained with Mayer's hematoxylin, dehydrated, mounted, and examined by light microscopy. For TNF-α immunostaining, the avidin biotin peroxidase method was used as previously described (23). The colonic sections were deparaffinized, rehydrated, and heated in a microwave oven in 0.01 M citrate buffer (pH 6.0) for 30 min. The sections were incubated at 4° C. overnight with anti-TNF (rabbit polyclonal IgG, Santa Cruz Biotechnology Inc., USA). This primary antibody was detected using avidin-biotin peroxidase detection solution (Dako), and the signal was visualized using diaminobenzidine. Slides were counterstained with Harris's hematoxylin, dehydrated, and then examined by light microscopy.

### Statistical Analysis

Data are reported as means±SE and were analyzed using SPSS version 22 (IBM, USA). One-way ANOVA and Tukey's post hoc tests were used to test differences among groups. P<0.05 was considered statistically significant.

### Results

### Disease Activity Score

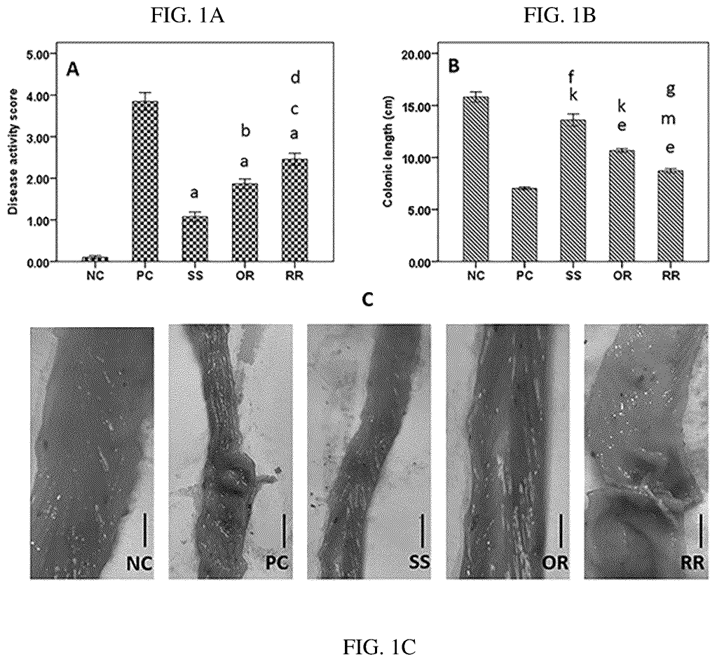

The TNBS-induced colitis rats showed an increased disease activity score compared with NC. All treatments significantly decreased this score. Sulfasalazine exerted the greatest decrease, and oral roflumilast decreased the score more than rectal roflumilast (FIG. 1A).

### Colonic Length and Macroscopic Appearance

TNBS-induced colitis rats showed shorter colonic length compared with NC rats, and all treatments significantly preserved the length. Sulfasalazine exerted the greatest effect, and oral roflumilast caused more improvement than rectal roflumilast (FIG. 1B). The appearance of the colon regarding inflammation, mucosal edema, erosions, and necrosis was evaluated. TNBS-induced colitis rats showed severe colonic lesions characterized by damaged mucosa and thickened bowel walls. The sulfasalazine group showed marked improvement, the oral roflumilast group showed moderate to marked improvement, and the rectal roflumilast group showed mild improvement (FIG. 1C).

### Serum Measurements

TNBS-induced colitis rats showed significantly increased serum levels of TNF-α, IL-2, and IL-6. All treatments significantly reversed these TNBS-induced changes. Sulfasalazine exerted the greatest decrease, and oral roflumilast decreased the levels more than rectal roflumilast (Table 1).

TABLE 1
The effects of roflumilast on serum levels of TNF-α, IL-2, and IL-6 in rats
with trinitrobenzenesulfonic acid (TNBS)-induced colitis (n = 8).

| | NC | PC | SS | OR | RR |
|---|---|---|---|---|---|
| TNF-α | 17.04 ± 0.67 | 91.18 ± 4.14 | 28.93 ± 0.5^{b, d} | 38.87 ± 2.33^{a, b, f} | 79.37 ± 1.88^{a, c, e} |
| (pg/mL) | | | | | |
| IL-2 | 196.10 ± 3.19 | 487.00 ± 9.36 | 257.97 ± 7.72^{g} | 302.75 ± 11.32^{g, h, i} | 363.87 ± 12.73^{g} |
| (pg/mL) | | | | | |
| IL-6 | 10.57 ± 0.42 | 47.04 ± 1.98 | 16.14 ± 1.13^{k, m} | 21.85 ± 1.00^{j, k, l} | 28.93 ± 0.50^{j, k} |
| (pg/mL) | | | | | |

Data are reported as means±SE; one-way ANOVA and Tukey's post hoc test. NC: normal control; PC: positive control; SS: sulfasalazine; OR: oral roflumilast; RR: rectal roflumilast. Tumor necrosis factor-α (TNF-α): ^{a}P<0.001 OR & RR vs NC; ^{b}P<0.001 SS & OR vs PC; ^{c}P<0.001 RR vs SS & OR; ^{d}P<0.01 SS vs NC (=0.008); ^{e}P<0.01 RR vs PC (=0.008); ^{f}P<0.05 OR vs SS (=0.034). Interleukin-2 (IL-2): ^{g}P<0.001 all treatments vs NC & PC; ^{h}P<0.01 OR vs RR (=0.001); ⁱP<0.05 OR vs SS (=0.016). Interleukin-6 (IL-6): ^{j}P<0.001 OR & RR vs NC; ^{k}P<0.001 all treatments vs PC; ^{l}P<0.01 OR vs RR (=0.001); ^{m}P<0.05 SS vs NC & OR (=0.013, 0.010).

### Colonic Measurements

TNBS-induced colitis rats showed significantly increased levels of MPO and MDA and significantly decreased GSH levels in the colon homogenates. All treatments significantly reversed these TNBS-induced changes. Sulfasalazine exerted the greatest changes, and oral roflumilast caused more changes than rectal roflumilast (Table 2).

TABLE 2
The effects of roflumilast on colonic levels of MPO, MDA, and GSH in
rats with trinitrobenzenesulfonic acid (TNBS)-induced colitis (n = 8).

| | NC | PC | SS | OR | RR |
|---|---|---|---|---|---|
| MPO activity | 11.29 ± 0.85 | 50.70 ± 1.05 | 18.38 ± 0.87^{a} | 23.89 ± 0.92^{a, b, c} | 28.17 ± 1.00^{a} |
| (μ/g) | | | | | |
| MDA | 19.31 ± 0.66 | 81.40 ± 2.13 | 28.93 ± 0.50^{e} | 36.68 ± 1.98^{d, e, f, g} | 47.04 ± 1.98^{d, e} |
| (mmol/mg) | | | | | |
| GSH | 19.60 ± 0.79 | 6.86 ± 0.22 | 14.73 ± 0.49^{h, i} | 11.94 ± 0.32^{h, i, k} | 9.24 ± 0.28^{h, j} |
| (mmol/mg) | | | | | |

Data are reported as means±SE; one-way ANOVA and Tukey's post hoc test. NC: normal control; PC: positive control; SS: sulfasalazine; OR: oral roflumilast; RR: rectal roflumilast. Myeloperoxidase (MPO): ^{a}P<0.001 all treatments vs NC & PC; ^{b}P<0.01 OR vs SS (=0.002); ^{c}P<0.05 OR vs RR (=0.022). Malonaldehyde (MDA): ^{d}P<0.001 OR & RR vs NC; ^{e}P<0.001 all treatments vs PC; ^{f}P<0.01 OR vs RR (=0.001); ^{g}P<0.05 OR vs SS (=0.014). Glutathione (GSH): ^{h}P<0.001 all treatments vs NC; ⁱP<0.001 SS & OR vs PC; ^{j}P<0.01 RR vs PC (=0.008); ^{k}P<0.01, OR vs SS & RR (=0.001, 0.002).

### Histopathological Examination

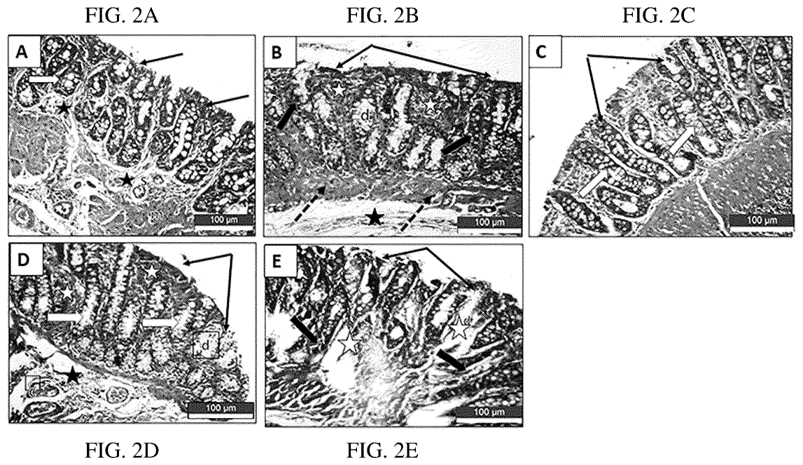

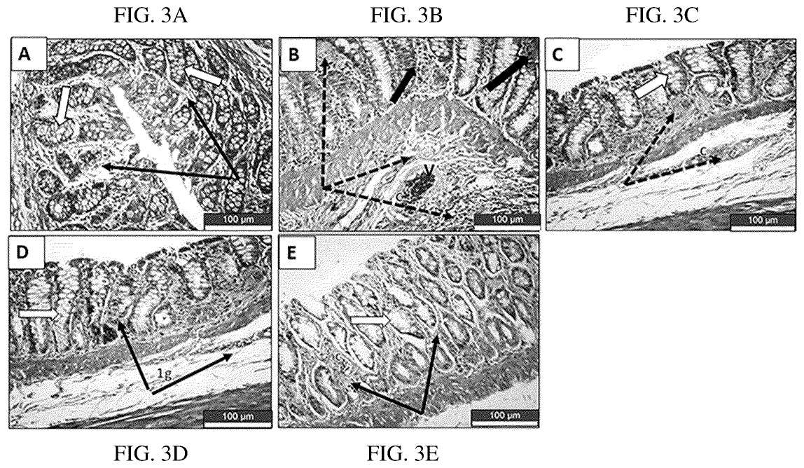

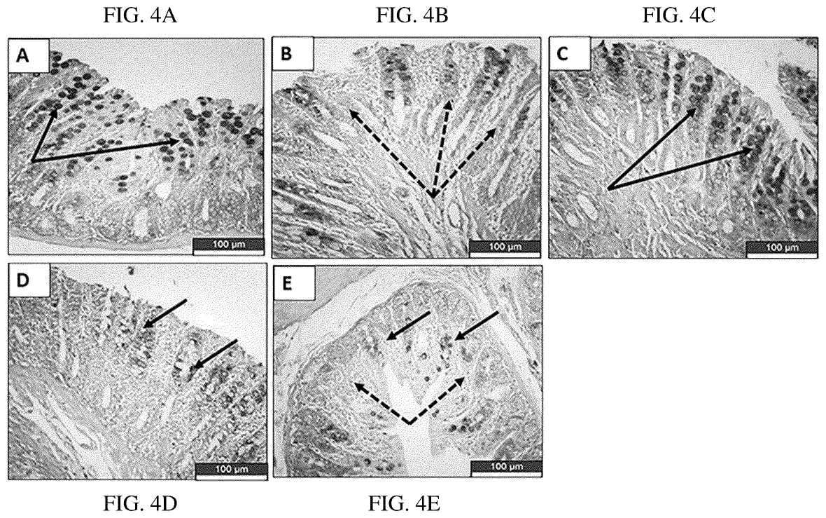

In the colonic sections stained with H&E, the TNBS-induced colitis rats exhibited destroyed glands, marked inflammatory infiltration, focal necrosis of mucosa and submucosa, loss of epithelial lining, and diffuse submucosal edema (FIG. 2). Masson trichrome staining for collagen and collagen fibers revealed that the TNBS group showed numerous congested blood vessels in the propria submucosa with edema around the connective tissue fibers and blood vessels. Lymphocytes accumulated in the propria submucosa, and some large nodules were located in the mucosa-submucosa (FIG. 3). PAS staining for mucopolysaccharides revealed that the TNBS group showed numerous goblet cells with positive PAS reactions that were prominent between the epithelial lining of the crypts. Some crypts of Lieberkühn showed ulceration and leucocyte accumulation (FIG. 4). All treatments exerted significant prophylactic effects, preventing the inflammatory changes to occur and interfering with the induction of chronic colitis by TNBS. Sulfasalazine exerted the greatest improvement, and oral roflumilast caused more improvement than rectal roflumilast.

### Immunohistochemical Results

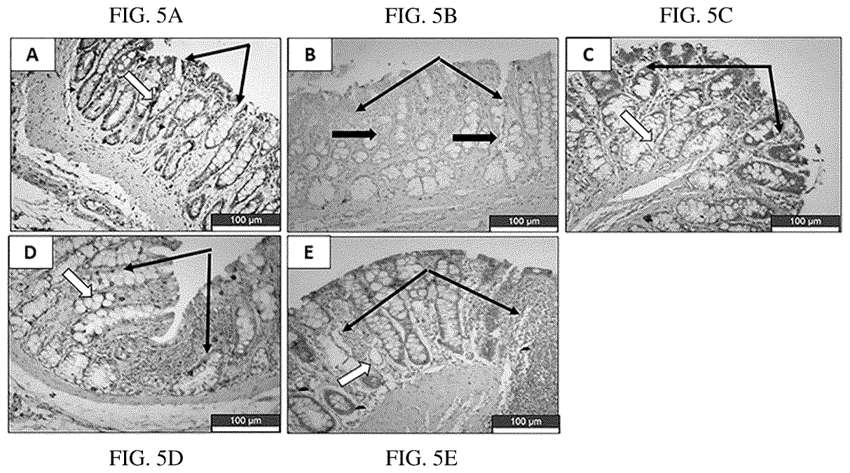

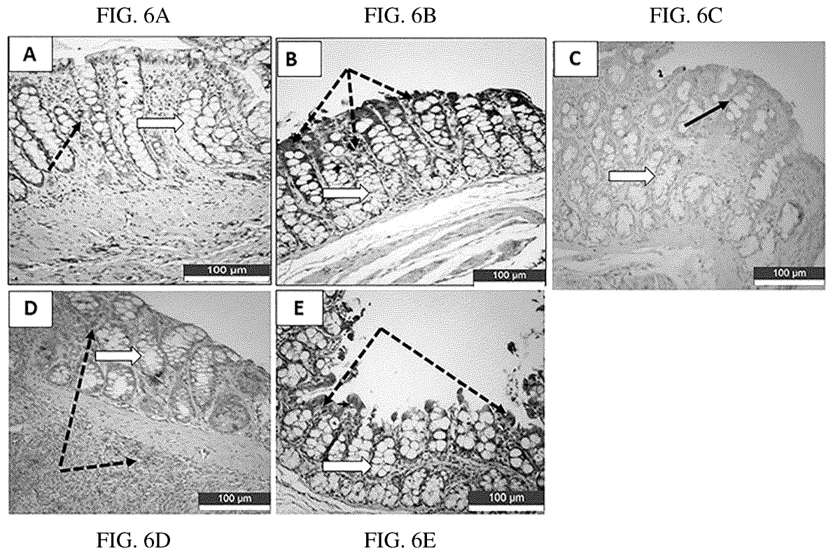

KI-67 staining revealed very faint positive reactions in the colonic sections of TNBS-induced colitis rats (FIG. 5). TNF-α staining revealed strong positive reactions in the TNBS group (FIG. 6). Both stains revealed that all the treatments reversed these TNBS-induced changes to different degrees. Sulfasalazine exerted the greatest improvement, and oral roflumilast caused more improvement than rectal roflumilast.

### Discussion

The chronic TNBS-induced colitis model is used to induce several CD-like features, such as transmural inflammation, Th1 immune pattern colitis, and fibrosis (4,12). A systematic review concluded that the chronic TNBS-induced colitis preclinical model can be acquired with several TNBS administrations and is most commonly characterized by the concentrations of inflammatory biomarkers, e.g., TNF-α and IL-6 (24). Inflammatory cytokines are crucial in immune responses, and their increased levels are related to the pathogenesis of IBD pathogenesis. The advantage of the chronic compared with the acute model is that the acute model only causes injury to the epithelium, leading to self-limiting inflammation rather than chronic disease (25). Additionally, in chronic TNBS-induced colitis, the parameters for histological evaluation of colonic sections stained with H&E included epithelial damage and inflammatory infiltration. TNBS-induced chronic colitis was characterized by submucosal edema, mucosal damage, and severe inflammatory infiltration. Cytokines, including IL-2, IL-6, and TNF-α, were measured in blood for cytokine profiling and to identify which cytokines best discriminate experimental colitis from controls (26). The histopathological evaluation of TNBS-induced chronic colitis included severity of inflammation and mucosal epithelial lesion; generally, diffuse transmural necrosis with hemorrhage was observed (24,27).

PDE4 is an intracellular enzyme that increases the production of inflammatory mediators and reduces the production of anti-inflammatory mediators. As such, it is implicated in the pathogenesis of many inflammatory diseases. PDE4 inactivates cyclic adenosine monophosphate and is the main PDE isoenzyme in mononuclear inflammatory cells, the principal source of TNF-α (6). It was reported that the increase in TNF-α plays a crucial role in the pathogenesis of IBD. IL-2 is another common cellular inflammatory factor. In inflammatory conditions, TNF-α and IL-2 levels rapidly increase and thus activate white blood cells, promote the migration of inflammatory cells, and expand the inflammatory response (28). Inhibition of PDE4 leads to widespread anti-inflammatory effects including decreased TNF-α levels. Thus, specific inhibition of PDE4 could be effective in treating numerous chronic inflammatory disorders (29). Interestingly, roflumilast is clinically effective at relatively small doses compared with the other PDE4 inhibitors. It improved episodic memory in subjects with minimal cognitive impairment at a low non-emetic dose with a plasma level nearly five-fold lower than the dose for chronic obstructive pulmonary disease patients. Using such low doses minimizes typical side effects of PDE4 inhibitors such as vomiting (30). Roflumilast showed a more potent anti-inflammatory activity in both animals and humans and was better tolerated than the preceding PDE4 inhibitors, e.g., rolipram and cilomilast (31).

The results of the current study are in agreement with a previous study that revealed that oral roflumilast (1 or 5 mg·kg⁻¹·day⁻¹) dose-dependently improved the disease clinical score (weight loss, stool consistency, and hemorrhage), colonic length, and colonic TNF-α production in mice with dextran sulphate sodium (DSS)-induced colitis (13). However, the histological score was not improved in the previous study (13). In addition, roflumilast showed potential anti-inflammatory effects in DSS-induced ulcerative colitis in male Wistar rats. Colitis was defined by assessing weight loss, colonic length, histological score, TNF-α levels, nitric oxide levels, cyclic adenosine monophosphate levels, MPO activity, and inducible nitric oxide synthase gene expression in colonic tissue. Roflumilast (5 mg/kg) diminished the severity of colitis, as it preserved colon length, decreased weight loss, and improved histological scores compared with the DSS group. It also decreased colon concentrations of TNF-α and NO, MPO activity, and inducible nitric oxide synthase gene expression. The effects of roflumilast were comparable to those exerted by sulfasalazine (32).

In the current study, TNBS-induced chronic colitis showed severe colonic injury characterized macroscopically by shortened colonic length, inflammation, mucosal edema, and thickening of the bowel wall. It was reported that TNBS-chronic colitis can be evaluated macroscopically with colon weight, length, wall thickness, and signs of inflammation, including hyperemia and mucosal edema (24). Colon length is considered an indirect measure of tissue integrity and severity of inflammation. TNBS-induced colitis decreases colon length, increases bowel thickness, and causes ulceration and hyperemia (33). Our results were in agreement with another study that reported that roflumilast partially reverses the TNBS-induced reduction in colon length at 1 and 5 mg-kg⁻¹·day⁻¹ and decreases the elevated colonic concentration of TNF-α (13).

In the current study, we investigated the effects of rectal roflumilast on TNBS-induced chronic colitis. Our results showed that rectal roflumilast reversed the TNBS-induced colitic changes, suggesting its local anti-inflammatory effects. A previous study on colitis-induced rats reported that oral roflumilast has local anti-inflammatory effects. Oral roflumilast dose-dependently improved the clinical score of colitis, reduced the colonic shortening, and decreased local TNFα expression in colonic tissue; however, this improvement was not correlated with a decreased histological score (13). Additionally, in a clinical trial, roflumilast cream applied topically once daily to affected areas of psoriasis was superior to vehicle cream in causing an almost clear state at 6 weeks. Thus, it seems that topical roflumilast has the potential to help existing therapies in many inflammatory skin diseases (34).

In the current study, the colonic sections of the TNBS-induced colitis group showed destroyed glands, marked inflammatory infiltration, numerous congested blood vessels with edema, loss of goblet cells, and ulceration in crypts. Roflumilast improved the TNBS-induced histopathological and immunohistochemical changes in rats. This is in agreement with a previous study that showed a close relationship between crypt injuries and clinical colitis activity (35), but contradicts another study that reported that roflumilast does not significantly change the histological score (13). The antigen KI-67 is a nuclear protein that is considered a marker of cellular proliferation. KI-67 is present during the active phases of the cell cycle and absent during the resting phase (36). In DSS-induced ulcerative colitis, epithelial apoptosis increased approximately five-fold, and the number of mitotic cells decreased by almost a half compared with the control group. The KI-67 immunohistochemical examination showed that crypt cells with cell cycle arrest at the resting stage increased nearly two-fold compared with the control group, indicating reduced proliferation. This might disrupt the epithelial barrier mechanism, facilitating mucosal invasion by intraluminal microorganisms (22). In TNBS-induced colitis, decreased KI-67 expression in colon epithelium indicates a smaller number of epithelial cells undergoing proliferation compared with the normal control. The number of KI-67-positive epithelial cells increased with treatment, implying regeneration of the injured epithelium (37). In TNBS-induced colitis, the increased TNF-α immunoreactivity in colonic tissue was significantly reduced by anti-inflammatory treatments in rats (38,39).

In conclusion, both oral and rectal roflumilast improved TNBS-induced chronic colitis, an experimental model resembling human CD, indicating local anti-inflammatory effects. Oral roflumilast caused more improvement than rectal roflumilast but was generally inferior to sulfasalazine, the standard treatment of CD. Nevertheless, our findings suggested that roflumilast could be used either via the oral or rectal route as an add-on off-label treatment for CD.

### REFERENCES

1. Ananthakrishnan A N. Epidemiology and risk factors for IBD. Nat Rev Gastroenterol Hepatol. 2015; 12:205-217. doi: 10.1038/nrgastro.2015.34.
2. Affronti A, Orlando A, Cottone M. An update on medical management on Crohn's disease. Expert Opin Pharmacother. 2015; 16:63-78. doi: 10.1517/14656566.2015.981525.
3. Goyal N, Rana A, Ahlawat A, Bijjem K R V, Kumar P. Animal models of inflammatory bowel disease: a review. Inflammopharmacology. 2014; 22:219-233. doi: 10.1007/s10787-014-0207-y.
4. Randhawa P K, Singh K, Singh N, Jaggi A S. A review on chemical-induced inflammatory bowel disease models in rodents. Korean J Physiol Pharmacol. 2014; 18:279-288. doi: 10.4196/kjpp.2014.18.4.279.
5. Murad H A, Habib H S, Rafeeq M M, Sulaiman M I, Abdulrahman A S, Khabaz M N. Co-inhalation of roflumilast, rather than formoterol, with fluticasone more effectively improves asthma in asthmatic mice. Exp Biol Med (Maywood) 2017; 242:516-526. doi: 10.1177/1535370216685006.
6. Bethke T D, Lahu G. High absolute bioavailability of the new oral phosphodiesterase-4 inhibitor roflumilast. Int J Clin Pharmacol Ther. 2011; 49:51-57. doi: 10.5414/CPP49051.
7. Hesslinger C, Schudt C, Marx D, Braun C. Combination of a Pd4 Inhibitor and a Tetrahydrobiopterin Derivative. Google Patents. 2008
8. Kohl B, Mueller B, Palosch W. Novel process for the preparation of roflumilast. Google Patents. 2009
9. Beume R, Hatzelmann A, Marx D, Schudt C, Tenor H, Eddahibi S, et al. Roflumilast for the treatment of pulmonary hypertension. Google Patents. 2006
10. Rehman N U, Ansari M N, Samad A. In silico, ex vivo and in vivo studies of roflumilast as a potential antidiarrheal and antispasmodic agent: inhibition of the PDE-4 enzyme and voltage-gated Ca++ ion channels. Molecules. 2020; 25:1008. doi: 10.3390/molecules25041008.
11. Saeedan A S, Rastogi S, Ansari M N. Roflumilast counteracts DMH-induced preneoplastic colon damage in albino Wistar rats. Hum Exp Toxicol. 2020; 39:1545-1555. doi: 10.1177/0960327120931165.
12. Loeuillard E, Bertrand J, Herranen A, Melchior C, Guérin C, Coëffier M, et al. 2, 4, 6-trinitrobenzene sulfonic acid-induced chronic colitis with fibrosis and modulation of TGF-β1 signaling. World J Gastroenterol. 2014; 20:18207-18215. doi: 10.3748/wjg.v20.i48.18207.
13. Rieder F, Siegmund B, Bundschuh D S, Lehr H A, Endres S, Eigler A. The selective phosphodiesterase 4 inhibitor roflumilast and phosphodiesterase 3/4 inhibitor pumafentrine reduce clinical score and TNF expression in experimental colitis in mice. Plos One. 2013; 8:e56867. doi: 10.1371/journal.pone.0056867.
14. El-Ashmawy N E, Khedr N F, El-Bahrawy H A, El-Adawy S A. Roflumilast, type 4 phosphodiesterase inhibitor, attenuates inflammation in rats with ulcerative colitis via down-regulation of iNOS and elevation of cAMP. Int Immunopharmacol. 2018; 56:36-42. doi: 10.1016/j.intimp 0.2018.01.004.
15. Al-Kishali H A, Abd El Fattah M A, Mohammad W A, El-Abhar H S. Cilostazol against 2, 4, 6-trinitrobenzene sulfonic acid-induced colitis: effect on tight junction, inflammation, and apoptosis. JGH Open. 2019; 3:281-289. doi: 10.1002/jgh3.12148.
16. Wang X, Yang J, Cao Q, Tang J. Therapeutic efficacy and mechanism of water-soluble extracts of Banxiaxiexin decoction on BALB/c mice with oxazolone-induced colitis. Exp Ther Med. 2014; 8:1201-1204. doi: 10.3892/etm.2014.1890.
17. Ghia J E, Blennerhassett P, Kumar-Ondiveeran H, Verdu E F, Collins S M. The vagus nerve: a tonic inhibitory influence associated with inflammatory bowel disease in a murine model. Gastroenterology. 2006; 131:1122-1130. doi: 10.1053/j.gastro.2006.08.016.
18. Day Y J, Marshall M A, Huang L, McDuffie M J, Okusa M D, Linden J. Protection from ischemic liver injury by activation of A2A adenosine receptors during reperfusion: inhibition of chemokine induction. Am J Physiol Gastrointest Liver Physiol. 2004; 286:G285-G293. doi: 10.1152/ajpgi.00348.2003.
19. Moron M S, Depierre J W, Mannervik B. Levels of glutathione, glutathione reductase and glutathione S-transferase activities in rat lung and liver. Biochim Biophys Acta. 1979; 582:67-78. doi: 10.1016/0304-4165(79)90289-7.
20. Ohkawa H, Ohishi N, Yagi K. Assay for lipid peroxides in animal tissues by thiobarbituric acid reaction. Anal Biochem. 1979; 95:351-358. doi: 10.1016/0003-2697(79)90738-3.
21. Wirtz S, Neufert C, Weigmann B, Neurath M F. Chemically induced mouse models of intestinal inflammation. Nat Protoc. 2007; 2:541-546. doi: 10.1038/nprot.2007.41.
22. Araki Y, Mukaisyo K, Sugihara H, Fujiyama Y, Hattori T. Increased apoptosis and decreased proliferation of colonic epithelium in dextran sulfate sodium-induced colitis in mice. Oncol Rep. 2010; 24:869-874. doi: 10.3892/or.2010.869.
23. Rajendiran V, Natarajan V, Devaraj S N. Anti-inflammatory activity of Alpinia officinarum hance on rat colon inflammation and tissue damage in DSS induced acute and chronic colitis models. Food Sci Hum Well. 2018; 7:273-281. doi: 10.1016/j.fshw.2018.10.004.
24. Silva I, Pinto R, Mateus V. Preclinical study in vivo for new pharmacological approaches in inflammatory bowel disease: a systematic review of chronic model of TNBS-induced colitis. J Clin Med. 2019; 8:1574. doi: 10.3390/jcm8101574.
25. Wirtz S, Popp V, Kindermann M, Gerlach K, Weigman B, Fichtner-Feigl S, et al. Chemically induced mouse models of acute and chronic intestinal inflammation. Nat Protoc. 2017; 12:1295-1309. doi: 10.1038/nprot.2017.044.
26. Alex P, Zachos N C, Nguyen T, Gonzales L, Chen T E, Conklin L S, et al. Distinct cytokine patterns identified from multiplex profiles of murine DSS and TNBS-induced colitis. Inflamm Bowel Dis. 2009; 15:341-352. doi: 10.1002/ibd.20753.
27. Motavallian-Naeini A, Andalib S, Rabbani M, Mahzouni P, Afsharipour M, Minaiyan M. Validation and optimization of experimental colitis induction in rats using 2, 4, 6-trinitrobenzene sulfonic acid. Res Pharm Sci. 2012; 7:159-169.
28. Oh Y, Kwon Y S, Jung B D. Anti-inflammatory effects of the natural compounds cortex phellodendri and Humulus japonicus on pelvic inflammatory disease in mice. Int J Med Sci. 2017; 14:729-734. doi: 10.7150/ijms.19616.
29. Li H, Zuo J, Tang W. Phosphodiesterase-4 inhibitors for the treatment of inflammatory diseases. Front Pharmacol. 2018; 9:1048. doi: 10.3389/fphar.2018.01048.
30. Sugin L J S, Murugesan A, Bindu M, Sunil K N. Roflumilast: a potential drug for the treatment of cognitive impairment? Neurosci Lett. 2020:135281. doi: 10.1016/j.neulet.2020.135281.
31. Bundschuh D S, Eltze M, Barsig J, Wollin L, Hatzelmann A, Beume R. In vivo efficacy in airway disease models of roflumilast, a novel orally active PDE4 inhibitor. J Pharmacol Exp Ther. 2001; 297:280-290.
32. El-Ashmawy N E, Khedr N F, El-Bahrawy H A, El-Adawy S A. Roflumilast, type 4 phosphodiesterase inhibitor, attenuates inflammation in rats with ulcerative colitis via down-regulation of iNOS and elevation of cAMP. Int Immunopharmacol. 2018; 56:36-42. doi: 10.1016/j.intimp 0.2018.01.004.
33. Antoniou E, Margonis G A, Angelou A, Pikouli A, Argiri P, karavokyros I, et al. The TNBS-induced colitis animal model: an overview. Ann Med Surg (Lond) 2016; 11:9-15. doi: 10.1016/j.amsu.2016.07.019.
34. Lebwohl M G, Papp K A, Gold L S, Gooderham M J, Kircik L H, Draelos Z D, et al. Trial of roflumilast cream for chronic plaque psoriasis. N Engl J Med. 2020; 383:229-239. doi: 10.1056/NEJMoa2000073.
35. Cooper H S, Murthy S N, Shah R S, Sedergran Dj. Clinicopathologic study of dextran sulfate sodium experimental murine colitis. Lab Invest. 1993; 69:238-249.
36. Cuylen S, Blaukopf C, Politi A Z, Müller-Reichert T, Neumann B, Poser I, et al. Ki-67 acts as a biological surfactant to disperse mitotic chromosomes. Nature. 2016; 535:308-312. doi: 10.1038/nature18610.
37. Jiang X G, Sun K, Liu Y Y, Yan L, Wang M X, Fan J Y, et al. Astragaloside IV ameliorates 2,4,6-trinitrobenzene sulfonic acid (TNBS)-induced colitis implicating regulation of energy metabolism. Sci Rep. 2017; 7:41832. doi: 10.1038/srep41832.
38. Toklu H Z, Kabasakal L, Imeryuz N, Kan B, Celikel C, Cetinel S, et al. A study comparing the efficacy of antimicrobial agents versus enzyme (P-gp) inducers in the treatment of 2,4,6 trinitrobenzenesulfonic acid-induced colitis in rats. J Physiol Pharmacol. 2013; 64:439-451.
39. Polat F R, Karabo\check{g}a I. Immunohistochemical examination of anti-inflammatory and anti-apoptotic effects of hesperetin on trinitrobenzene sulfonic acid induced colitis in rats. Biotech Histochem. 2019; 94:151-158. doi: 10.1080/10520295.2018.1530454.
40. Antoniou E, Margonis G A, Angelou A, Pikouli A, Argiri P, Karavokyros I, et al. The TNBS-induced colitis animal model: an overview. Ann Med Surg. 2016; 11:9-15. doi: 10.1016/j.amsu.2016.07.019.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A method of ameliorating inflammation associated with Crohn's Disease in a subject in need thereof, comprising administering rectally to the subject a therapeutically effective amount of a composition comprising roflumilast wherein the therapeutically effective amount is an amount sufficient to ameliorate inflammation in a digestive tract of the subject.

2. The method of claim 1, wherein the composition is administered repetitively over a period of days.

3. The method of claim 2, wherein the composition is administered once daily for 1-30 days.

4. The method of claim 1, wherein the therapeutically effective amount is 1-10 mg/kg.

5. The method of claim 1, wherein the composition contains or is co-administered with one or more agents different from roflumilast, wherein said one or more agents are directed against bowel disease.

6. The method of claim 5, wherein the one or more agents includes a phosphodiesterase IV inhibitor.

7. The method of claim 5, wherein the one or more agents includes sulfasalazine.

8. A suppository dosage form, comprising roflumilast and a pharmaceutically acceptable carrier.
